(19) United States
(12) Patent Application Publication
(10) Pub. No.: US 2022/0354411 A1
(43) Pub. Date: Nov. 10, 2022

(54) NATURAL MOVEMENT EEG RECOGNITION METHOD BASED ON SOURCE LOCALIZATION AND BRAIN NETWORKS

(71) Applicant: SOUTHEAST UNIVERSITY, Nanjing City (CN)

(72) Inventors: Baoguo XU, Nanjing City, Jiangsu (CN); Leying DENG, Nanjing City, Jiangsu (CN); Yifei WANG, Nanjing City, Jiangsu (CN); Xin WANG, Nanjing City, Jiangsu (CN); Aiguo SONG, Nanjing City, Jiangsu (CN)

(21) Appl. No.: 17/634,418

(22) PCT Filed: Nov. 30, 2020
(86) PCT No.: PCT/CN2020/132582

§ 371 Date: Feb. 10, 2022

(30) Foreign Application Priority Data
Nov. 11, 2020 (CN) 202011255263.9

(51) Int. Cl. A61B 5/372; A61B 5/00

(52) U.S. Cl. CPC A61B 5/372; A61B 5/7267; A61B 5/7203; A61B 5/7278; A61B 5/725; G06F 3/015

(57) ABSTRACT

Disclosed is a natural movement electroencephalogram (EEG) recognition method based on source localization and a brain network, which includes the following steps: (1) performing multi-channel EEG measurement for natural movements; (2) preprocessing acquired EEG signals, and extracting the movement-related cortical potential (MRCP), and θ, α, β, and γ rhythms; (3) determining a lead field matrix of the signals, calculating initial solutions of sources by means of L1 regularization constraint, and then performing iteration by means of successive over-relaxation to obtain a source localization result; (4) by using the sources as nodes, calculating PLV between each pair of sources at each time point by means of short-time sliding window, and establishing brain networks; and (5) calculating a network adjacency matrix at each time point and five brain network indicators, introducing these features into a classifier for training and testing, and conducting a statistical test for the brain network indicators. The present disclosure makes improvements to the conventional source localization method by using the T-wMNE algorithm in combination with successive over-relaxation, and establishes brain networks by using the sources as nodes, thus improving the EEG decoding accuracy for natural movements and revealing the neural mechanism of the human body.

### TECHNICAL FIELD

The present disclosure belongs to the field of biological signal processing; and relates to an electroencephalogram (EEG) signal recognition method, and more particularly to a natural movement EEG recognition method based on source localization and brain networks, which can provide technical means for EEG decoding of natural movements.

### BACKGROUND

Brain-computer interface (BCI) is a research hotspot in the field of rehabilitation engineering and neural engineering in recent years, which provides a means to directly communicate with the outside world through EEG signals. For the past few years, BCI-based rehabilitation training has heavily relied on repetitive imagination of basic motor tasks. For example, repetitive imagination of foot movement is used as the control signal for holding the glass, which brings the user an unnatural and uncoordinated operating experience. In order to give the user a better operating experience, the imagined movement should be as close as possible to the actual movement. However, the natural movements, such as palmar, pinch, twist, plug, etc., are relatively complicated and many joints are used, and these movements usually activate the similar brain motor area, so that the conventional EEG recognition methods cannot make a good distinction between these natural movements.

EEG source localization and brain network analysis is one of the research hotspots in the BCI field in recent years. The source localization includes source feature reconstruction and source location determination, and the brain network can establish functional or causal connections between nodes. The study on the signals of natural movements at the source level helps to overcome the volume conduction effect, thus improving the decoding accuracy. Further, the brain network constructed with the sources can help to reveal the neural mechanism of the human body.

### SUMMARY

To solve the foregoing problems, the present disclosure discloses a natural movement EEG recognition method based on source localization and brain networks, which can decode EEG signals of natural movements by means of source localization and constructing brain networks with the sources.

To achieve the foregoing objective, the present disclosure adopts the following technical solution: A natural movement EEG recognition method based on source localization and brain networks is provided, which includes the following steps:

(1) performing multi-channel EEG measurement for natural movements;
(2) preprocessing acquired EEG signals, removing artefacts, and extracting the movement-related cortical potential (MRCP), θ rhythm, α rhythm, β rhythm and γ rhythm;
(3) determining a lead field matrix of the signals, and calculating initial solutions of sources by means of L1 regularization constraint; and then performing iteration for the initial solutions by means of successive over-relaxation, and using the latest solution vector as a final estimation result of source localization after iteration completion;
(4) by using the sources as nodes, calculating phase locking value (PLV) between each pair of sources at each time point by means of short-time sliding window; and when the PLV is greater than a set threshold, constructing an edge between the two sources, and using a standardized value of PLV as the weight of the edge; and
(5) calculating the characteristic path length, clustering coefficient, average node strength, average betweenness, efficiency, and network adjacency matrix at each time point; introducing these features into a classifier for training and testing; and conducting a statistical test for the first 5 features, to analyze differences in time or frequency of these features corresponding to different movements.

Step (2) includes the following sub-steps:

(a1) performing pre-filtering for the acquired EEG signals;
(a2) eliminating the data channel with abnormal kurtosis and performing spherical interpolation, which uses an average value of four channels closest to the interpolated channel as a value of this channel;
(a3) identifying and removing electrooculogram (EOG) and electromyogram (EMG) components from the EEG by means of blind source separation algorithm;
(a4) extracting epochs and correcting baseline for the EEG;
(a5) removing trials with absolute value of amplitude greater than 200 μV, abnormal joint probability or abnormal kurtosis, where the thresholds of the latter two are 5 times the standard deviation of their statistic;
(a6) performing common average reference (CAR) for the EEG; and
(a7) performing zero-phase Butterworth bandpass filtering at 0.3 Hz to 3 Hz, 4 Hz to 8 Hz, 8 Hz to 13 Hz, 13 Hz to 30 Hz, and 30 Hz to 45 Hz separately for the EEG after re-reference, and extracting the MRCP, θ rhythm, α rhythm, β rhythm, and γ rhythm.

Step (3) includes the following sub-steps:

(b1) selecting a head model;
(b2) solving a forward problem, to obtain the lead field matrix L;
(b3) determining a time point to be analyzed, and setting an iteration error ε and the maximum number K of iterations;

(b4) calculating an initial solution of a source vector by means of the T-wMNE algorithm:

sₜ⁽⁰⁾=min ∥vₜ−Lsₜ∥₂+λ∥Wsₜ∥₁

where W=diag(∥l₁∥,∥l₂∥, . . . ,∥l_{N}∥) is a weighted matrix; N is the number of the sources, which is equal to the number of electrodes herein; sₜ denotes the source vector at the time point t; vₜ denotes the electrode potential at the time point t; and λ is the regularization coefficient;
(b5) performing iteration for the initial solution obtained in sub-step (b4) by means of successive over-relaxation:

$$\{ \begin{matrix}\begin{matrix}\begin{matrix}{s}_{1 , t}^{( k + 1 )} = ( 1 - ω ) {s}_{1 , t}^{( k )} + w ( {v}_{1 , t} - {l}_{1 , 2} {s}_{2 , t}^{( k )} - {l}_{1 , 3} {s}_{3 , t}^{( k )} - … - {l}_{1 , N} {s}_{N , t}^{( k )} ) / {l}_{1 , 1} \\ {s}_{2 , t}^{( k + 1 )} = ( 1 - ω ) {s}_{2 , t}^{( k )} + w ( {v}_{2 , t} - {l}_{2 , 1} {s}_{1 , t}^{( k + 1 )} - {l}_{2 , 3} {s}_{3 , t}^{( k )} - … - {l}_{2 , N} {s}_{N , t}^{( k )} ) / {l}_{2 , 2}\end{matrix} \\ ⋮\end{matrix} \\ \begin{matrix}{s}_{N , t}^{( k + 1 )} = ( 1 - ω ) {s}_{N , t}^{( k )} + \\ w ( {v}_{N , t} - {l}_{N , 1} {s}_{1 , t}^{( k + 1 )} - {l}_{N , 2} {s}_{2 , t}^{( k + 1 )} - … - {l}_{N , N - 1} {s}_{N - 1 , t}^{( k + 1 )} ) / {l}_{N , N}\end{matrix}\end{matrix}$$

where s_{i,t} denotes a value of the ith source at the time point t, and i=1,2, . . . , N; v_{j,t} denotes the potential of the jth electrode at the time point t, and j=1, 2, . . . , N; ω is a relaxation factor; and k denotes the number of iterations; and
(b6) when ∥sₜ^{(k+1)}−sₜ^{(k)}∥≤ε or k>K, the iteration ends, and the latest solution vector is used as the final estimation result of source localization; otherwise, continuing the iteration.

Particularly, in sub-step (b5), with 0.01 step size between (1, 2), ω that minimizes ∥vₜ−Lsₜ∥ after 10 iterations is selected as the optimal value.

In step (4), Hilbert transform is performed on the source vector of a single trial at each time point, to obtain the phase of the source vector at each time point; and then the PLV of each pair of sources at each time point is calculated:

$$\hat{{s}_{l}} ( t ) = H ( {s}_{i} ( t ) ) = {\int }_{- ∞}^{+ ∞} \frac{{s}_{i} ( τ )}{π ( t - τ )} d τ$$

$$ϕ ( t ) = arctan ( \frac{\hat{{s}_{l}} ( t )}{{s}_{i} ( t )} )$$

$${Δϕ}_{ij} = {ϕ}_{i} - {ϕ}_{j}$$

$${PLV}_{ij} = \frac{1}{M} \vert {\sum }_{m = 1}^{M} {e}^{j Δ {ϕ}_{ij}} \vert$$

where m=1, 2, . . . , M, which denotes the mth trial; and
when PLVᵢⱼ is greater than the threshold, an edge between sources i and j is constructed; and the PLV is subjected to standardization processing and the standardized value is used as the weight of the edge:

$${w}_{ij} = \frac{{PLV}_{ij} - min ( PLV )}{max ( PLV ) - min ( PLV )}$$

In step (5), the statistical test method is t-test; and the classifier is the sLDA classifier, which is trained and tested by performing five-fold cross-validation for ten times.

The present disclosure has the following beneficial effects:

(1) The present disclosure performs EEG source localization for natural movements by using the T-wMNE algorithm in combination with successive over-relaxation, which, compared to the conventional source localization method, improves the solution accuracy and speed while ensuring sparsity of the solution and robustness of the calculation process.

(2) The present disclosure establishes brain networks by using the sources as nodes. Compared to the conventional method of establishing the brain network by using electrode channels as the nodes, the method of the present disclosure can intuitively show the differences in the dynamic change process of the sources corresponding to different natural movements, thus revealing the neural mechanism of the human body.

(3) The present disclosure performs source localization and brain network analysis separately at different frequency bands, so that the differences in activation of the different natural movements in different frequency bands and the root cause of the MRCP's discrimination between different natural movements can be intuitively shown.

### BRIEF DESCRIPTION OF THE DRAWINGS

FIG. 1 is a flowchart of a natural movement EEG recognition method based on source localization and brain networks of the present disclosure;

FIG. 2 is a flowchart of EEG signal preprocessing in the natural movement EEG recognition method based on source localization and brain networks of the present disclosure; and

FIG. 3 is a flowchart of EEG source localization in the natural movement EEG recognition method based on source localization and brain networks of the present disclosure.

### DETAILED DESCRIPTION OF THE EMBODIMENTS

The present disclosure is further elaborated below with reference to the accompanying drawings and a specific embodiment. It should be noted that the following specific implementation is merely used to describe the present disclosure rather than limiting the scope of the present disclosure.

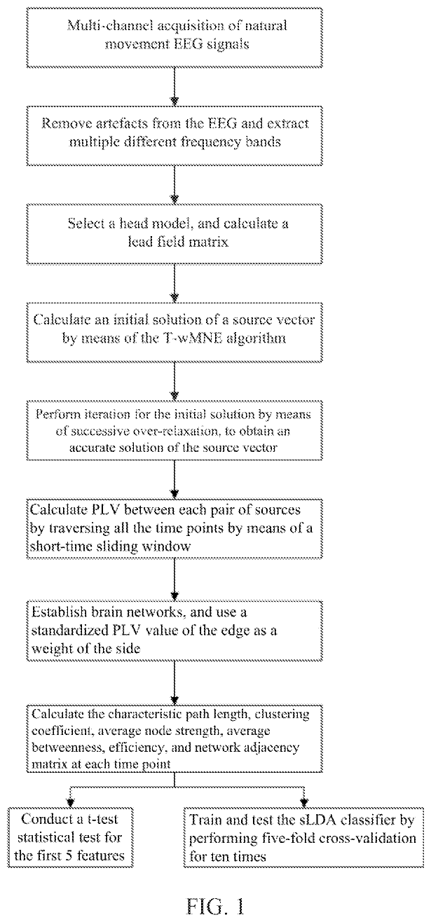

The present disclosure designs a natural movement EEG recognition method based on source localization and brain networks, which, as shown in FIG. 1, includes the following steps:

(1) performing multi-channel EEG measurement for natural movements;
(2) preprocessing acquired EEG signals, removing artefacts, and extracting the MRCP, θ rhythm, α rhythm, β rhythm, and γ rhythm ;
(3) determining a lead field matrix of the signals, and calculating initial solutions of sources by means of L1 regularization constraint; and then performing iteration for the initial solutions by means of successive over-relaxation, and using the latest solution vector as a final estimation result of source localization after iteration completion;
(4) by using the sources as nodes, calculating PLV between each pair of sources at each time point by means of short-time sliding window; and when the PLV is greater than a set threshold, constructing an edge between the two sources, and using a standardized value of PLV as the weight of the edge; and
(5) calculating the characteristic path length, clustering coefficient, average node strength, average betweenness, efficiency, and network adjacency matrix at each time point; introducing these features into a classifier for training and testing; and conducting a statistical test for the first 5 features, to analyze differences in time or frequency of these features corresponding to different movements.

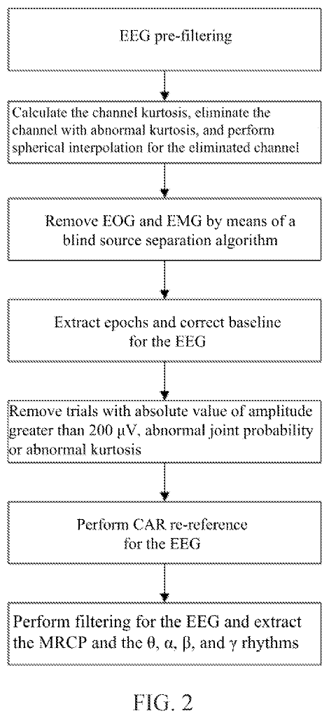

As shown in FIG. 2, step (2) includes the following sub-steps:

(a1) performing pre-filtering for the acquired EEG signals;
(a2) eliminating the data channel with abnormal kurtosis and performing spherical interpolation, which uses an average value of four channels closest to the interpolated channel as a value of this channel;
(a3) identifying and removing EOG and EMG components from the EEG by means of a blind source separation algorithm;
(a4) extracting epochs and correcting baseline for the EEG;
(a5) removing trials with absolute value of amplitude greater than 200 μV, abnormal joint probability or abnormal kurtosis, where the thresholds of the latter two are 5 times the standard deviation of their statistic;
(a6) performing CAR for the EEG; and
(a7) performing zero-phase Butterworth bandpass filtering at 0.3 Hz to 3 Hz, 4 Hz to 8 Hz, 8 Hz to 13 Hz, 13 Hz to 30 Hz, and 30 Hz to 45 Hz separately for the EEG after re-reference, and extracting the MRCP, θ rhythm, α rhythm, β rhythm, and γ rhythm.

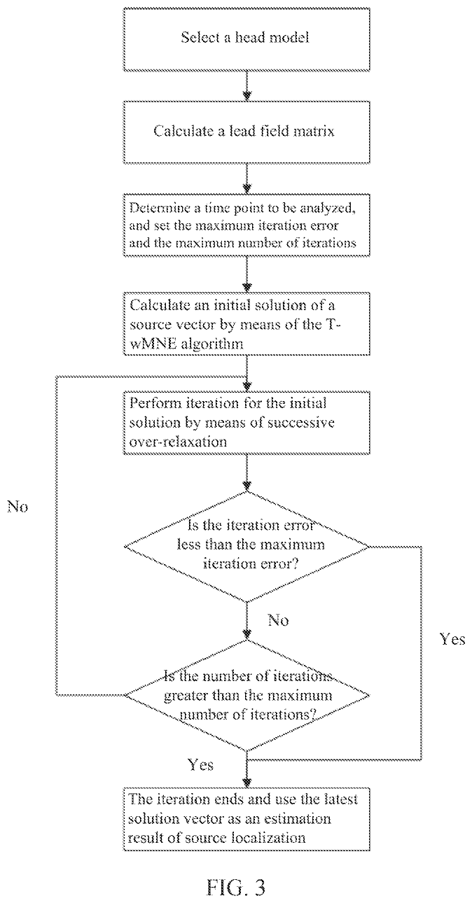

As shown in FIG. 3, step (3) includes the following sub-steps:

(b1) selecting a head model;
(b2) dividing the scalp into N small 3D grids; placing three current dipoles with the dipole moment along X, Y, and Z axes in each grid, where their vector sum is equivalent to a possible current dipole; and determining the lead field matrix L according to the following equation:

$$V = [ \begin{matrix}v ( {r}_{1} , 1 ) & ⋯ & v ( {r}_{1} , T ) \\ ⋮ & ⋱ & ⋮ \\ v ( {r}_{N} , 1 ) & ⋯ & v ( {r}_{N} , T )\end{matrix} ]$$

$$= [ \begin{matrix}l ( {r}_{1} , {r}_{1}^{*} ) {e}_{1} & ⋯ & l ( {r}_{1} , {r}_{N}^{*} ) {e}_{N} \\ ⋮ & ⋱ & ⋮ \\ l ( {r}_{N} , {r}_{1}^{*} ) {e}_{1} & ⋯ & l ( {r}_{N} , {r}_{N}^{*} ) {e}_{N}\end{matrix} ] [ \begin{matrix}{s}_{1 , 1} & ⋯ & {s}_{1 , T} \\ ⋮ & ⋱ & ⋮ \\ {s}_{N , 1} & ⋯ & {s}_{N , T}\end{matrix} ]$$

$$= LS$$

where the ith column in L denotes a potential distribution generated by the ith current dipole source at each electrode position; rᵢ* denotes a position vector of the current dipole; rⱼ denotes a position vector of the measured scalp electrode; s=seᵢ denotes the dipole moment (s is the size and eᵢ is the direction) of the current dipole; v(rⱼ, T) denotes the potential of the jth electrode at the time point t; i=1, . . . , N, which denotes that there are N current dipoles; and j=1, . . . , N, which denotes that there are N measurement electrodes;
(b3) determining a time point to be analyzed, and setting an iteration error ε and the maximum number K of iterations;
(b4) calculating an initial solution of a source vector by means of the T-wMNE algorithm:

sₜ⁽⁰⁾=min ∥vₜ−Lsₜ∥₂+λ∥Wsₜ∥₁

where W=diag(∥l₁∥,∥l₂∥, . . . ,∥l_{N}∥) is a weighted matrix; N is the number of the sources, which is equal to the number of electrodes herein; sₜ denotes the source vector at the time point t; vₜ denotes the electrode potential at the time point t; and λ is the regularization coefficient;
(b5) performing iteration for the initial solution obtained in sub-step (b4) by means of successive over-relaxation:

$$\{ \begin{matrix}\begin{matrix}\begin{matrix}{s}_{1 , t}^{( k + 1 )} = ( 1 - ω ) {s}_{1 , t}^{( k )} + w ( {v}_{1 , t} - {l}_{1 , 2} {s}_{2 , t}^{( k )} - {l}_{1 , 3} {s}_{3 , t}^{( k )} - … - {l}_{1 , N} {s}_{N , t}^{( k )} ) / {l}_{1 , 1} \\ {s}_{2 , t}^{( k + 1 )} = ( 1 - ω ) {s}_{2 , t}^{( k )} + w ( {v}_{2 , t} - {l}_{2 , 1} {s}_{1 , t}^{( k + 1 )} - {l}_{2 , 3} {s}_{3 , t}^{( k )} - … - {l}_{2 , N} {s}_{N , t}^{( k )} ) / {l}_{2 , 2}\end{matrix} \\ ⋮\end{matrix} \\ \begin{matrix}{s}_{N , t}^{( k + 1 )} = ( 1 - ω ) {s}_{N , t}^{( k )} + \\ w ( {v}_{N , t} - {l}_{N , 1} {s}_{1 , t}^{( k + 1 )} - {l}_{N , 2} {s}_{2 , t}^{( k + 1 )} - … - {l}_{N , N - 1} {s}_{N - 1 , t}^{( k + 1 )} ) / {l}_{N , N}\end{matrix}\end{matrix}$$

where ω∈(1,2) is a relaxation factor, and ω that minimizes ∥vₜ−Lsₜ∥ after 10 iterations is selected as the optimal relaxation factor with 0.01 step size between (1, 2); s_{i,t} denotes a value of the ith source at the time point t, and i=1,2, . . . , N; v_{j,t} denotes the potential of the jth electrode at the time point t, and j=1, 2, . . . , N; and k denotes the number of iterations; and
(b6) when ∥sₜ^{(k+1)}−sₜ^{(k)}∥≤ε or k>K, the iteration ends, and the latest solution vector is used as the final localization estimation result of the source; otherwise, continuing the iteration.

In step (4), Hilbert transform is performed on the source vector of a single trial at each time point, to obtain the phase of the source vector at each time point; and then the PLV of each pair of sources at each time point is calculated:

$$\hat{{s}_{l}} ( t ) = H ( {s}_{i} ( t ) ) = {\int }_{- ∞}^{+ ∞} \frac{{s}_{i} ( τ )}{π ( t - τ )} d τ$$

$$ϕ ( t ) = arctan ( \frac{\hat{{s}_{l}} ( t )}{{s}_{i} ( t )} )$$

$${Δϕ}_{ij} = {ϕ}_{i} - {ϕ}_{j}$$

$${PLV}_{ij} = \frac{1}{M} \vert {\sum }_{m = 1}^{M} {e}^{j Δ {ϕ}_{ij}} \vert$$

where m=1, 2, . . . , M, which denotes the mth trial.

When PLVᵢⱼ is greater than the set threshold, an edge between sources i and j is constructed; and the PLV is subjected to standardization processing and the standardized value is used as the weight of the edge:

$${w}_{ij} = \frac{{PLV}_{ij} - min ( PLV )}{max ( PLV ) - min ( PLV )}$$

In step (5), the statistical test method is t-test; and the classifier is the sLDA classifier, which is trained and tested by performing five-fold cross-validation for ten times.

What is claimed is:

1. A natural movement electroencephalogram (EEG) recognition method based on source localization and a brain network, comprising the following steps:
(1) performing multi-channel EEG measurement for natural movements;
(2) preprocessing acquired EEG signals, removing artefacts, and extracting the movement-related cortical potential (MRCP) , θ rhythm, α rhythm, β rhythm, and γ rhythm;
(3) determining a lead field matrix of the signals, and calculating initial solutions of sources by means of L1 regularization constraint; and then performing iteration for the initial solutions by means of successive over-relaxation, and using the latest solution vector as a final estimation result of source localization after iteration completion;
(4) by using the sources as nodes, calculating PLV between each pair of sources at each time point by means of short-time sliding window; and when the PLV is greater than a set threshold, constructing an edge between the two sources, and using a standardized value of PLV as the weight of the edge; and
(5) calculating the characteristic path length, clustering coefficient, average node strength, average betweenness, efficiency, and network adjacency matrix at each time point;
introducing these features into a classifier for training and testing; and conducting a statistical test for the first 5 features, to analyze differences in time or frequency of these features corresponding to different movements.

2. The natural movement EEG recognition method based on source localization and a brain network according to claim 1, wherein step (2) comprises the following sub-steps:
(a1) performing pre-filtering for the acquired EEG signals;
(a2) eliminating the data channel with abnormal kurtosis and performing spherical interpolation, which uses an average value of four channels closest to the interpolated channel as a value of this channel;
(a3) identifying and removing EOG and EMG components from the EEG by means of a blind source separation algorithm;
(a4) extracting epochs and correcting baseline for the EEG;
(a5) removing trials with absolute value of amplitude greater than 200 μV, abnormal joint probability or abnormal kurtosis, wherein the thresholds of the latter two are 5 times the standard deviation of their statistic;
(a6) performing common average reference (CAR) for the EEG; and
(a7) performing zero-phase Butterworth bandpass filtering at 0.3 Hz to 3 Hz, 4 Hz to 8 Hz, 8 Hz to 13 Hz, 13 Hz to 30 Hz, and 30 Hz to 45 Hz separately for the EEG after re-reference, and extracting the MRCP and the θ, α, β, and γ rhythms.

3. The natural movement EEG recognition method based on source localization and a brain network according to claim 1, wherein step (3) comprises the following sub-steps:
(b1) selecting a head model;
(b2) solving a forward problem, to obtain the lead field matrix L;
(b3) determining a time point to be analyzed, and setting an iteration error ε and the maximum number K of iterations;
(b4) calculating an initial solution of a source vector by means of the T-wMNE algorithm:
sₜ⁽⁰⁾=min∥vₜ−Lsₜ∥₂+λ∥Wsₜ∥₁
wherein W=diag(∥l₁∥,∥₂∥, . . . ,∥l_{N}∥) is a weighted matrix; N is the number of the sources, which is equal to the number of electrodes herein; sₜ denotes the source vector at the time point t; vₜ denotes the electrode potential at the time point t; and λ is the regularization coefficient;
(b5) performing iteration for the initial solution obtained in sub-step (b4) by means of successive over-relaxation:
$$\{ \begin{matrix}\begin{matrix}\begin{matrix}{s}_{1 , t}^{( k + 1 )} = ( 1 - ω ) {s}_{1 , t}^{( k )} + w ( {v}_{1 , t} - {l}_{1 , 2} {s}_{2 , t}^{( k )} - {l}_{1 , 3} {s}_{3 , t}^{( k )} - … - {l}_{1 , N} {s}_{N , t}^{( k )} ) / {l}_{1 , 1} \\ {s}_{2 , t}^{( k + 1 )} = ( 1 - ω ) {s}_{2 , t}^{( k )} + w ( {v}_{2 , t} - {l}_{2 , 1} {s}_{1 , t}^{( k + 1 )} - {l}_{2 , 3} {s}_{3 , t}^{( k )} - … - {l}_{2 , N} {s}_{N , t}^{( k )} ) / {l}_{2 , 2}\end{matrix} \\ ⋮\end{matrix} \\ \begin{matrix}{s}_{N , t}^{( k + 1 )} = ( 1 - ω ) {s}_{N , t}^{( k )} + \\ w ( {v}_{N , t} - {l}_{N , 1} {s}_{1 , t}^{( k + 1 )} - {l}_{N , 2} {s}_{2 , t}^{( k + 1 )} - … - {l}_{N , N - 1} {s}_{N - 1 , t}^{( k + 1 )} ) / {l}_{N , N}\end{matrix}\end{matrix}$$
wherein s_{i,t} denotes a value of the ith source at the time point t, and i=1,2, . . . , N; v_{j,t} denotes the potential of the jth electrode at the time point t, and j=1, 2, . . . , N; ω is a relaxation factor; and k denotes the number of iterations; and
(b6) when ∥sₜ^{(k+1)}−sₜ^{(k)}∥≤ε or k>K, the iteration ends, and the latest solution vector is used as the final estimation result of source localization; otherwise, continuing the iteration.

4. The natural movement EEG recognition method based on source localization and a brain network according to claim 1, wherein in step (4), Hilbert transform is performed on the source vector of a single trial at each time point, to obtain the phase of the source vector at each time point; and then the PLV of each pair of sources at each time point is calculated:
$$\hat{{s}_{l}} ( t ) = H ( {s}_{i} ( t ) ) = {\int }_{- ∞}^{+ ∞} \frac{{s}_{i} ( τ )}{π ( t - τ )} d τ$$
$$ϕ ( t ) = arctan ( \frac{\hat{{s}_{l}} ( t )}{{s}_{i} ( t )} )$$
$${Δϕ}_{ij} = {ϕ}_{i} - {ϕ}_{j}$$
$${PLV}_{ij} = \frac{1}{M} \vert {\sum }_{m = 1}^{M} {e}^{j Δ {ϕ}_{ij}} \vert$$
wherein m=1, 2, . . . , M, which denotes the mth trial; and
when PLVᵢⱼ is greater than the threshold, an edge between sources i and j is constructed;
and the PLV is subjected to standardization processing and the standardized value is used as the weight of the edge:
$${w}_{ij} = \frac{P L {V}_{ij} - min ( PLV )}{max ( P L V ) - min ( P L V )}$$

5. The natural movement EEG recognition method based on source localization and a brain network according to claim 1, wherein in step (5), the statistical test method is t-test;
and the classifier is the sLDA classifier, which is trained and tested by performing five-fold cross-validation for ten times.

6. The natural movement EEG recognition method based on source localization and a brain network according to claim 3, wherein in sub-step (b5), with 0.01 step size between (1, 2), ω that minimizes ∥vₜ−Lsₜ∥ after 10 iterations is selected as the optimal relaxation factor.
